(19) United States
(12) Patent Application Publication
(10) Pub. No.: US 2013/0268000 A1
(43) Pub. Date: Oct. 10, 2013

(54) TISSUE GRAFT ANCHORING

(71) Applicants: Christopher D. Harner, Pittsburgh, PA (US); Michael Charles Ferragamo, Foster, RI (US); Michael James Perriello, Hopedale, MA (US); Alfred Rodrigue Berube, JR., North Attleboro, MA (US)

(72) Inventors: Christopher D. Harner, Pittsburgh, PA (US); Michael Charles Ferragamo, Foster, RI (US); Michael James Perriello, Hopedale, MA (US); Alfred Rodrigue Berube, JR., North Attleboro, MA (US)

(21) Appl. No.: 13/803,909

(22) Filed: Mar. 14, 2013

Related U.S. Application Data
(60) Provisional application No. 61/614,878, filed on Mar. 23, 2012.

(51) Int. Cl. A61F 2/08; A61B 17/04

(52) U.S. Cl. CPC A61F 2/0811; A61B 17/0401
USPC 606/232

(57) ABSTRACT

A fixation device includes a member, a woven material attached to the member, and suture extending through the woven material and including free regions for securing the woven material to a tissue graft. A method of securing a tissue graft includes providing a fixation member having a woven material attached thereto and suture extending through the woven material; determining a desired distance between the fixation member and a tissue graft; and attaching the woven material to the tissue graft using the suture at a position to obtain the desired distance.

### CROSS-REFERENCE TO RELATED APPLICATIONS

The present application claims priority from U.S. Provisional Application Ser. No. 61/614,878, filed Mar. 23, 2012, titled “TISSUE GRAFT ANCHORING,” the contents of which are incorporated herein by reference in their entirety.

### TECHNICAL FIELD

This document relates to tissue graft anchoring.

### BACKGROUND

An anterior cruciate ligament (ACL) that has ruptured and is non-repairable is generally replaced arthroscopically by a tissue graft. The tissue graft can be harvested from a portion of a tendon/bone reconstruction tissue, i.e., Achilles tendon; patellar; or quadriceps tendon having so called “bone blocks” on at least one end, and from the semitendinosus and gracilis. These can be harvested as autographs or allographs. Alternatively, the tissue graft can be formed from synthetic materials or from a combination of synthetic and natural materials.

The replacement tissue graft is implanted by securing one end of the tissue graft in a socket formed in a passage within the femur, and passing the other end of the graft through a passage formed in the tibia. Generally, sutures are used to affix each end of the tissue graft to a fastener (e.g., an interference screw or a post), which is then secured to the bone.

It is also known to use a graft fixation member, e.g., a fixation button, to secure the tissue graft at the femoral cortex, as described in U.S. Pat. No. 5,306,301 (“the '301 patent”) hereby incorporated by reference in its entirety. When using a fixation button, the femoral passage generally includes a relatively larger diameter portion for receiving the graft, and a smaller diameter, passing channel near the femoral cortex for receiving a length of suture that runs from the tissue graft to the fixation button. By measuring the total length of the femoral passage and the length of the larger diameter portion of the femoral passage, the surgeon determines the appropriate length of suture material for attaching the fixation button to the tissue graft.

### SUMMARY

According to one aspect, a fixation device includes a member, a woven material attached to the member, and suture extending through the woven material and including free regions for securing the woven material to a tissue graft.

According to another aspect, a method of securing a tissue graft includes providing a fixation member having a woven material attached thereto and suture extending through the woven material, determining a desired distance between the fixation member and a graft ligament, and attaching the woven material to the tissue graft using the suture at a position to obtain the desired distance.

Implementations of either of these aspects may include one or more of the following features. For example, the member may define at least one opening. The member may be elongated in a first dimension defining a length that extends between first and second ends of the member, and the member may have a second dimension transverse to the first dimension that is smaller than the length.

The woven material may include a Chinese finger trap. The woven material may be attached to the member by direct fixation of the woven material through the openings or may be attached to the member by fibers that are fed through the openings and woven through the woven material. Needles may be attached to the suture. Attaching the woven material to the tissue graft using the suture may include attaching the woven material using the needles attached to the suture

The woven material may be in the form of a “V” shape and configured to be sutured to the tissue graft.

The woven material may be in the form of two lengths of material that are attached at one end to the member and may be configured to be sewn together around the tissue graft. Attaching the woven material to the tissue graft may include sewing the two lengths of material together around the tissue graft.

A tubular element defining a slot may be attached to the member and the woven material may form an open mesh around the tubular element. Attaching the woven material to the tissue graft using the suture may include attaching the woven material and tubular element to the tissue graft using needles attached to the suture.

The woven material may be about 30 mm long and 10 mm in diameter.

The details of one or more implementations are set forth in the accompanying drawings and the description below. Other features, objects, and advantages of the disclosure will be apparent from the description and drawings, and from the claims.

### BRIEF DESCRIPTION OF THE DRAWINGS

FIG. 1 illustrates a tissue graft secured within the knee by a graft fixation member.

FIG. 2 is another view of the knee joint of FIG. 1.

FIG. 3 is a perspective view of a fixation device.

FIGS. 4A-4C are side, end and perspective views of a fixation device.

FIGS. 5A and 5B are side and perspective views of an additional embodiment of a fixation device.

FIG. 6 is a perspective view of an additional embodiment of a fixation device.

### DETAILED DESCRIPTION

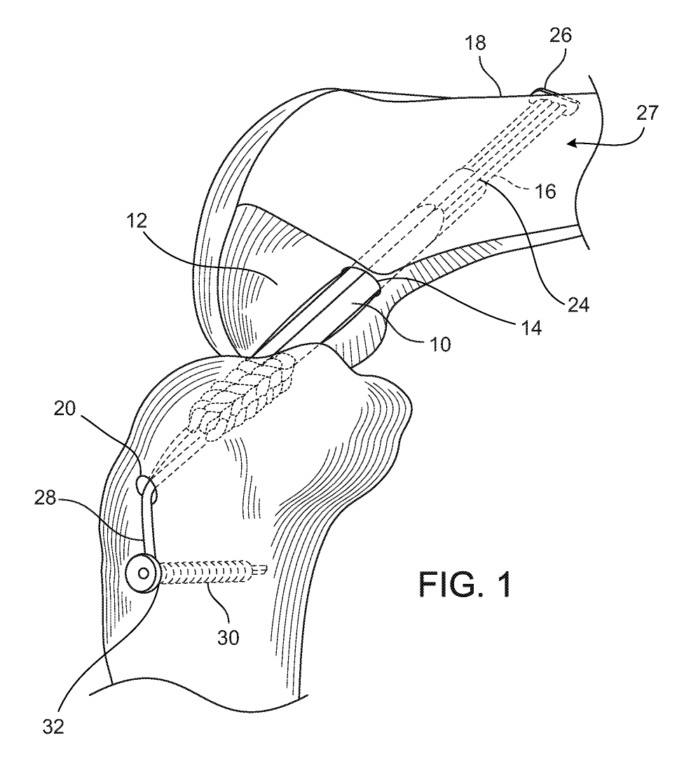

Referring to FIG. 1, a knee joint has a tissue graft 10 (for example, a semitendinosus and gracilis graft) implanted in an anterior cruciate ligament (ACL) repair and reconstruction procedure. Prior to implanting tissue graft 10, a femoral channel 14 for receiving one end of tissue graft 10 is then drilled from notch 12 a predetermined distance within the femur with a passing channel 16 of reduced diameter drilled further through the femur from femoral channel 14 to a region of femoral cortex 18. A tibial channel 20 for receiving the other end of tissue graft 10 is drilled from an anterior region of the tibia to a region near the opening of femoral channel 14.

The tissue graft 10 is secured at femoral cortex region 18 with a graft fixation member 26 of a fixation device 27. The tissue graft 10 is connected to the graft fixation member 26 by a woven construct 24, discussed below. The other end of tissue graft 10 includes a length of suture 28 which is attached to the tibia, for example, with a fixation screw 30. A washer 32 either attached to or positioned under the head of fixation screw 30 helps in holding the suture in place when screw 30 is tightened.

The graft fixation member 26 is positioned using pull threads (not shown) attached to the member. The pull threads are passed through the channels 14 and 16 from the notch 12 to the cortex 18 and used to pull the graft fixation member 26 through the channels 14 and 16 with a long axis of the graft fixation member aligned with the channels. After exiting the channel 16, the pull threads are used to flip the graft fixation member 26 so that the member 26 lies flat against the cortex.

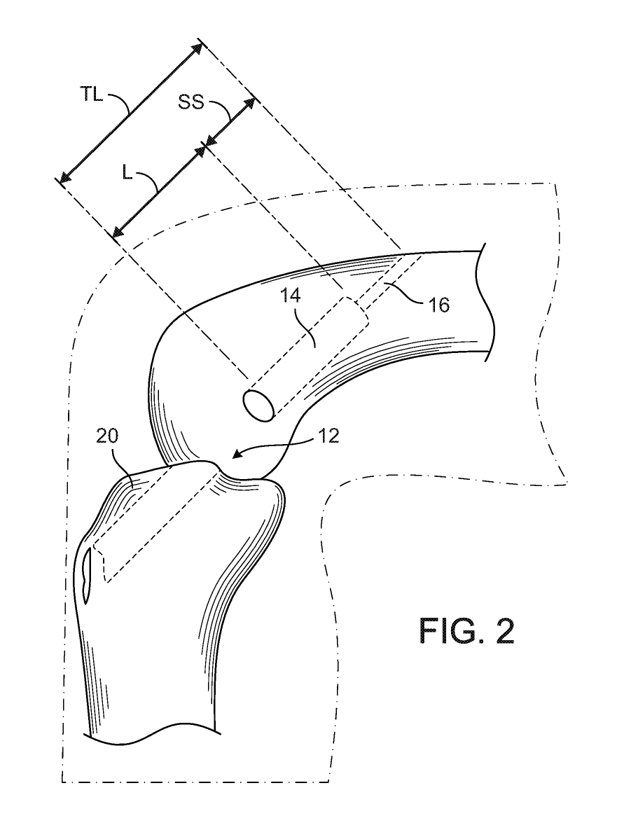

Referring to FIG. 2, length (L) of femoral channel 14 is selected by the surgeon in accordance with the desired insertion distance of tissue graft 10 within the femur. The additional span of construct 24, designated as SS, is approximately that of passing channel 16 so that the sum of the desired insertion length (L) and span of the construct (SS) is the measured total length (TL) from the opening of femoral channel 14 to the opening at femoral cortex 18. Each of these dimensions is measured prior to implanting the tissue graft so that during the implantation procedure, the surgeon, under arthroscopic observation, can be assured that tissue graft 10 has been properly positioned within femoral channel 14.

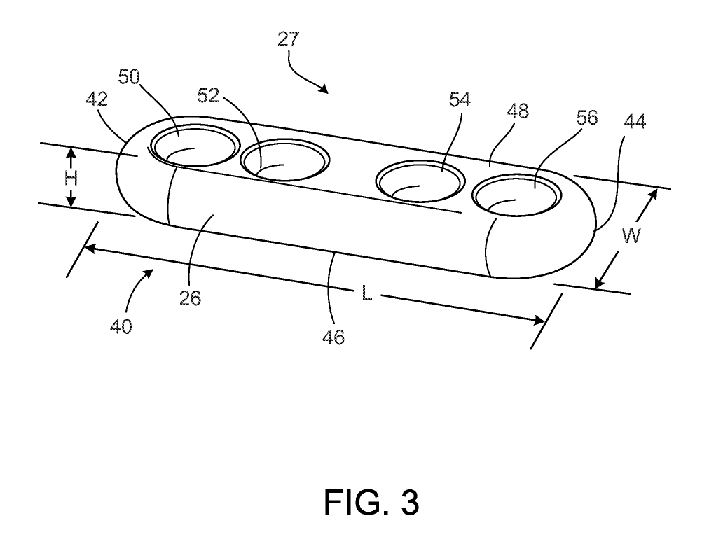

Referring to FIG. 3, the graft fixation member 26 is elongated in a first dimension defining a length, L, that extends between a first end 42 and a second end 44 of the member 26, a second dimension transverse to the first dimension that has a width, W, smaller than the length, L, and a third dimension transverse to the first and second dimensions that has a height, H, small than the length, L. The graft fixation member has a bone contacting side 46 and an opposite side 48. Extending between the sides 46, 48 are four holes 50, 52, 54 and 56. The holes 50 and 56 receive the passing sutures, not shown.

The length, width and height of the fixation member 26, is for example, 12 mm×4 mm×1.5 mm.

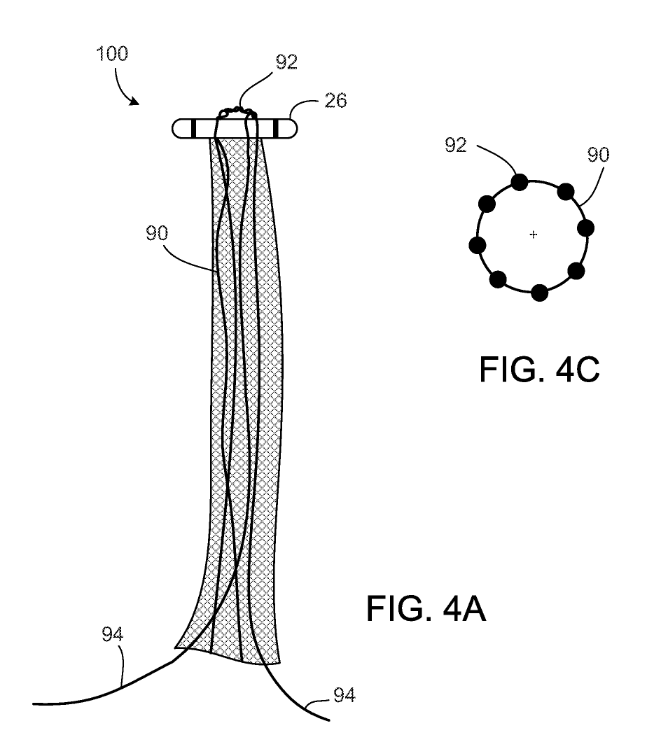

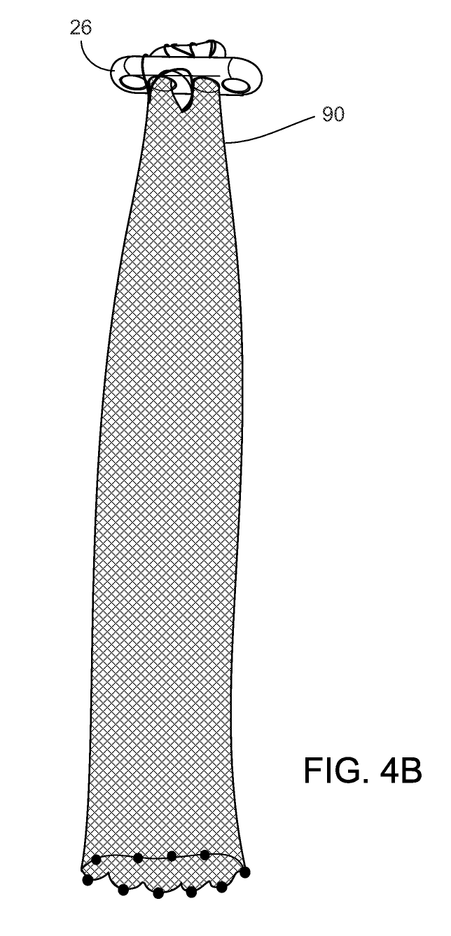

Referring to FIGS. 4A-4C, a fixation device 100 includes a woven or braided construct 24, for example, a Chinese Finger Trap 90 to hold the tissue graft in place at a predetermined distance from the graft fixation member 26. The predetermined distance ranges from 0 mm up to the length of the finger trap, for example, about 30 mm. The distance between the graft fixation member 26 and the tissue graft is determined by the surgeon prior to placing the graft, and set by loading the Chinese Finger Trap 90 onto the tissue graft to create the desired distance.

The finger trap is attached to the graft fixation member 26 by either direct fixation of the finger trap through the button through holes or by additional fibers 92 which are fed through the button through holes and then woven through the length of the finger trap. As shown in FIG. 4A, the fibers 92 can extend out of the base of the Chinese Finger Trap 90 with needles 94 attached so that the fibers can be stitched back through the Chinese Finger Trap 90 and the tissue graft once the tissue graft is positioned in the Chinese Finger Trap 90 prior to implanting the construct. The Chinese Finger Trap 90 is, for example, about 30 mm long and 10 mm in diameter. The fibers 92 can be woven or braided suture, for example, #5 Ultrabraid™ suture

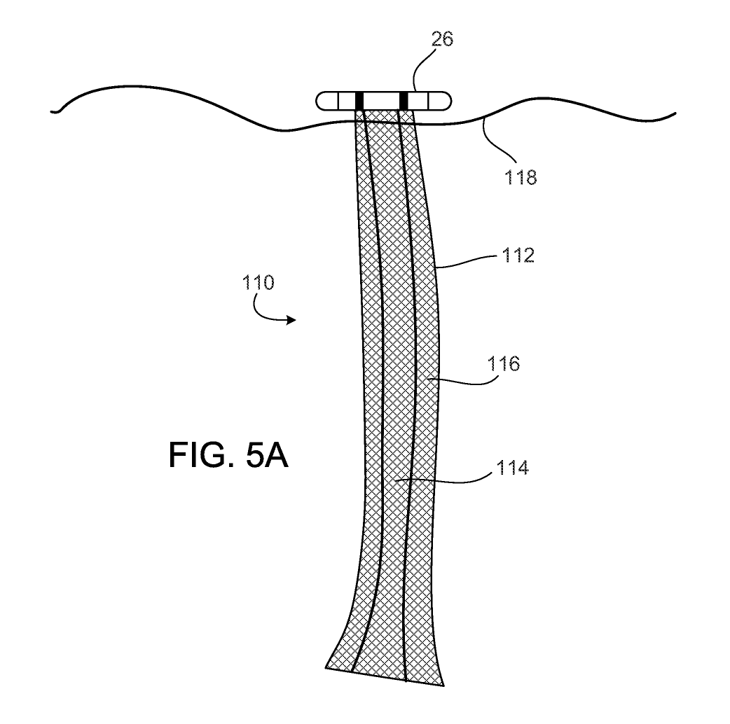

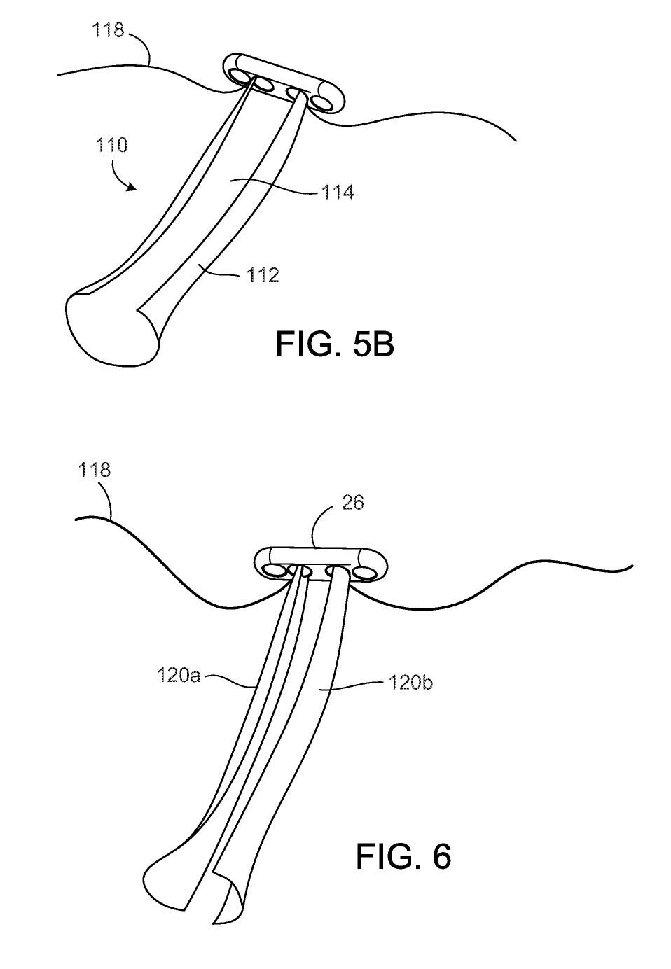

Rather than a Chinese Finger Trap 90, a stiff woven material can be made into a hot dog bun or extruded “V” shape and sutured to the tissue graft at the desired location. The stiff woven material can also be in the form of two lengths of material that attached at one end to the fixation device and sewn together around the tendon. Referring to FIGS. 5A and 5B, a fixation device 110 includes a tubular element 112 defining a slot 114. The tubular element 112 is formed of a stiff woven material, for example, polyethylene fiber. In use the tissue graft is placed within the tubular element 112 and the tubular element 112 is sutured to the tissue graft at a desired location. The tubular element 112 is surrounded by an open mesh 116 (not shown in FIG. 5B), and a suture 118 having needles (not shown) at its ends is weaved through the open mesh 116 and secured to the graft fixation member 26. The needled suture 118 is used by the operating room personnel to suture the tubular element 112 to the tissue graft. In FIG. 6, the tubular element 112 has been replaced by two, tubular sections 120a, 120b that receive a tissue graft therebetween.

Other implementations are within the scope of the following claims.

What is claimed is:

1. A fixation device comprising:
a member defining at least two openings, the member being elongated in a first dimension defining a length that extends between first and second ends of the member, the member having a second dimension transverse to the first dimension that is smaller than the length;
a woven material attached to the member; and
suture extending through the woven material and including free regions for securing the woven material to a tissue graft.

2. The fixation device of claim 1 wherein the woven material comprises a Chinese finger trap.

3. The fixation device of claim 1 wherein the woven material is attached to the member by direct fixation of the woven material through the openings.

4. The fixation device of claim 1 wherein the woven material is attached to the member by fibers that are fed through the openings and woven through the woven material.

5. The fixation device of claim 1 wherein needles are attached to the free regions of the suture.

6. The fixation device of claim 1 wherein the woven material is in the form of a “V” shape and configured to be sutured to the tissue graft.

7. The fixation device of claim 1 wherein the woven material is in the form of two lengths of material that are attached at one end to the member and configured to be sewn together around the tissue graft.

8. The fixation device of claim 1 further comprising a tubular element defining a slot and attached to the member.

9. The fixation device of claim 8 wherein the woven material forms an open mesh around the tubular element.

10. The fixation device of claim 9 wherein needles are attached to the free regions of the suture.

11. The fixation device of claim 1 wherein the woven material is about 30 mm long and 10 mm in diameter.

12. A method of securing a tissue graft comprising:
providing a fixation member having a woven material attached thereto and suture extending through the woven material;
determining a desired distance between the fixation member and a tissue graft; and
attaching the woven material to the tissue graft using the suture at a position to obtain the desired distance.

13. The method of claim 12 wherein needles are attached to the suture and attaching the woven material to the tissue graft using the suture includes attaching the woven material to the tissue graft using the needles attached to the suture.

14. The method of claim 12 wherein the woven material comprises a Chinese finger trap.

15. The method of claim 12 wherein the woven material is attached to the member by direct fixation of the woven material through the openings.

16. The method of claim 12 wherein the woven material is attached to the member by fibers that are fed through the openings and woven through the woven material.

17. The method device of claim 12 wherein the woven material is in the form of two lengths of material that are attached at one end to the member and attaching the woven material to the tissue graft includes sewing the two lengths of material together around the tissue graft.

18. The method of claim 12 wherein a tubular element defining a slot is attached to the fixation member.

19. The method of claim 18 wherein the woven material forms an open mesh around the tubular element.

20. The method of claim 19 wherein needles are attached to the suture and attaching the woven material to the tissue graft using the suture includes attaching the woven material and tubular element to the tissue graft using the needles attached to the suture.
